(19) United States
(12) Patent Application Publication
(10) Pub. No.: US 2016/0051320 A1
(43) Pub. Date: Feb. 25, 2016

(54) ELECTROSURGICAL INSTRUMENT WITH SELECTIVE CONTROL OF ELECTRODE ACTIVITY

(71) Applicant: HS West Investments, LLC, Sandy, UT (US)

(72) Inventor: Hugh S. West, JR., Sandy, UT (US)

(21) Appl. No.: 14/726,277

(22) Filed: May 29, 2015

Related U.S. Application Data
(63) Continuation-in-part of application No. 14/467,645, filed on Aug. 25, 2014.

(51) Int. Cl. A61B 18/14

(52) U.S. Cl. CPC A61B 18/1482; A61B 2018/00607

(57) ABSTRACT

Electrosurgical instruments are configured to provide increased ablative capability without requiring increased current density at the electrode. The electrosurgical instrument includes an elongate probe having a handle portion and a distal end. An electrode is disposed at the distal end and is configured to ablate tissue. The instrument includes an aspiration lumen, e.g., that may open through the electrode, at the distal end to aspirate fluid, tissue debris, and gaseous bubbles through the aspiration lumen. The electrosurgical instrument includes a user operable control (e.g., button) on the handle portion for selectively placing the instrument in boosted ablation mode, which can be achieved by restricting aspiration of fluid through the aspiration lumen, reducing active cooling of the electrode, and causing increased ablative sparking density at the electrode (e.g., by at least 10%, 20%, 35%, or 50%).

### CROSS REFERENCE TO RELATED APPLICATION

This application is a continuation-in-part of U.S. patent application Ser. No. 14/467,645, filed Aug. 25, 2014, the disclosure of which is incorporated herein in its entirety.

### BACKGROUND OF THE INVENTION

1. The Field of the Invention

The present invention relates to electrosurgical instruments which provide for selective increased ablative capability (e.g., increasing the size of the sparking ablative field (a discharge field) provided by the electrode) when desired, without requiring any increase in provided electrical power, as well as related methods of use.

2. the Relevant Technology

Electrosurgical procedures utilize an electrosurgical generator to supply radio frequency (RF) electrical power to an active electrode for ablating (i.e., vaporizing) and/or coagulating tissue. An electrosurgical probe is generally formed of a metallic conductor surrounded by a dielectric insulator such as plastic, ceramic, or glass. The electrode remains exposed and provides the surface which coagulates or ablates adjacent tissue. During an electrosurgical procedure, the metal electrode is often immersed in a conductive fluid and is brought in contact with or in close proximity to the tissue structure to be ablated or coagulated. During a procedure, the electrode is typically energized at a voltage of a few hundred to a few thousand volts and at a frequency between 100 kHz to over 4 MHz. The voltage induces a current in the conductive liquid and surrounding tissue. The most intense heating occurs in the region very close to the electrode where the current density is highest.

Depending on how the electrosurgical instrument is configured and how much power is provided, the heat generated from the device can be used to coagulate (e.g., cauterize) tissue or alternatively to ablate tissue. To cause ablation, the electrode generates enough heat to form gas bubbles around the electrode. The gas bubbles have a much higher resistance than tissue or saline, which causes the voltage across the electrode to increase. Given sufficient power, the electrode discharges (i.e., arcs). The high voltage current travels through the gas bubbles and creates a plasma discharge. This phenomenon visibly manifests itself in the conductive fluid medium as a sparking energy field adjacent the electrode. Where this occurs with the electrode close to the tissue, the generated plasma ablates the tissue.

Electrosurgical instruments can also be used for coagulating tissue. In coagulation, the current density at the electrode is configured to cause heating, but not tissue vaporization. The current density is generally lower than that provided during ablation, but is kept sufficiently high to cause proteins and/or other components of the tissue to agglomerate, thereby causing coagulation.

### BRIEF SUMMARY OF THE INVENTION

The present invention is directed to electrosurgical instruments for ablating tissue at a surgical site of a patient in a surgical procedure. These instruments typically have an aspiration means through the electrode to draw bubbles and debris to keep the field of view optically clear. One of the effects of aspiration, whether desired or not, is that fluid can pass by or through the electrode and actively draw heat away from and cool the electrode. Recognizing the active cooling aspect of fluid aspiration, the inventive instruments advantageously provide for a selective increase or boost in ablative capability by temporarily decreasing or stopping fluid aspiration, effectively increasing the size and/or density of the ablative sparking energy field (the discharge field) generated adjacent to the electrode. This boosted ablation mode can be entered when desired by a practitioner without increasing the current density at the electrode, which typically requires increasing the electrical power provided to the electrode or reducing the surface area of the electrode. Instead, boosted ablation is provided by selectively decreasing or stopping the flow of cooling fluid through an aspiration lumen adjacent to the electrode, that opens in the electrode.

An exemplary electrosurgical instrument may include an elongate probe having a handle portion, a distal end, and at least one electrode disposed at the distal end of the probe configured to perform ablation. An aspiration lumen may advantageously be disposed longitudinally through an interior of the elongate probe. The aspiration lumen may include an opening at the distal end of the probe so as to actively aspirate fluid (e.g., saline, tissue, and gaseous bubbles at a surgical site). Aspirating fluid through the lumen also provides active cooling of the electrode and/or fluid immediately adjacent to the electrode, whether desired or not, which has the effect of decreasing the strength of the ablative discharge.

A user operable control component for selectively restricting aspiration to the aspiration lumen is provided and can be disposed on the handle portion of the instrument for convenience. Such a control component temporarily slows or stops active suctioning of fluid through the aspiration lumen opening, which reduces or stops the flow of cooling liquid past the electrode. The result is reduced cooling and increased heat buildup at the electrode surface and adjacent fluid, which causes increased vaporization of water (e.g., more vapor bubbles) immediately adjacent to the electrode surface. This, in turn, increases electrical resistance at the electrode surface and surrounding fluid and increases sparking density at the electrode surface, which further increases heat at the electrode surface and surrounding tissue and boosts tissue ablation.

In many cases, the increase in sparking density at the electrode can visibly increase compared to the sparking density at the electrode when the instrument operates in a normal ablation mode, where active suctioning of fluid is provided. By way of example, the sparking density at the electrode when the electrosurgical instrument is placed into boosted ablation mode by decreasing or stopping aspiration of fluids through the aspiration lumen may increase by at least about 10% compared to the sparking density at the electrode during normal ablation mode (i.e., when aspiration of fluids is at the normal flow rate for the device or procedure). Preferably, the sparking density at the electrode is at least about 20% greater in boosted ablation mode, more preferably at least about 35% greater, and most preferably at least about 50% greater than when in normal ablation mode.

The heat produced adjacent to the electrode when in boosted ablation mode may similarly increase and, in many cases, may cause an increase in water vapor production as a percentage of normal water vapor production than the increase in sparking density. For example, the volume of water vapor bubbles produced by the electrode in boosted ablation mode may increase by at least about 20% compared to the amount of water vapor bubbles produced by the electrode in normal ablation mode. Preferably, the volume of water vapor bubbles produced by the electrode is at least about 40% greater in boosted ablation mode, more preferably at least about 70% greater, and most preferably at least about 100% greater than when in normal ablation mode.

Increased water vapor bubble production is often beneficial because the increase in heat and water vapor bubble production at the electrode correlates with the rate of tissue ablation. Accordingly, an increase in sparking density at the electrode, coupled with an increase in water vapor bubble production can correlate with an increase in the rate of tissue ablation. In some embodiments, the rate of tissue ablation in boosted ablation mode can be increased by at least about 10% compared to normal ablation mode. Preferably the rate of tissue ablation is increased by at least about 20%, more preferably by at least about 35%, and most preferably by at least about 50% in boosted ablation mode compared to normal tissue ablation mode.

In one embodiment, controls (e.g., one or more buttons) may be provided on the handle portion of the instrument for activating the electrode to operate in normal ablation mode and for selectively causing the electrode to operate in boosted ablation mode. The one or more controls may also cause the electrode to operate in coagulation mode (e.g., by reducing current density at the electrode to coagulate instead of ablate tissue). In one embodiment, decreasing or stopping the flow of aspirating fluid may cause the electrode to switch from coagulation mode to ablation mode. Because such controls (e.g., one or more buttons) are disposed on the handle portion, they are easily accessible to the practitioner's thumb (or fingers) of the hand that grips the instrument, without requiring the practitioner to release or adjust his or her grip.

Upon selection of the boosted ablation mode, active cooling of the electrode by aspirating fluid past the electrode is temporarily decreased, stopped, or simply not initiated. As a result of the reduction in active cooling when a boosted ablation mode is selected, fluid and tissue adjacent to the electrode are rapidly heated, providing a nearly instantaneous boost to ablative capability provided by the instrument. According to one embodiment, a control can be configured to only place the device in boosted ablation mode while the control component (e.g., button) is depressed or otherwise activated. Release of the control advantageously restores the device to the normal ablation mode and/or a coagulation mode. Such boosted ablation may be visibly manifested as an ablative sparking energy field of increased density and/or size relative to the density and/or size of the sparking energy field when in normal ablation mode.

Another aspect is a method of using the disclosed electrosurgical devices. Such method may include providing an instrument as described above, activating a control component disposed on the handle portion to place the device in normal ablation mode, and selectively activating a control component to temporarily place the device in boosted ablation mode. During normal ablation mode, a desired amount of power is supplied to the electrode and a desired amount of aspirating fluid is aspirated through the aspiration lumen adjacent to the electrode. During boosted ablation mode, activation of a control temporarily decreases or stops aspiration of fluids through the aspiration lumen, causing increased heat, increased vapor production (e.g., water bubbles) adjacent to the electrode, increased sparking density, and even more heat at the electrode, which further boosts the rate of tissue ablation. In some cases, there will be a visible increase in sparking density and light intensity at the electrode.

Further features and advantages of the present invention will become apparent to those of ordinary skill in the art in view of the detailed description of preferred embodiments below.

### BRIEF DESCRIPTION OF THE DRAWINGS

To further clarify the above and other advantages and features of the present invention, a more particular description of the invention will be rendered by reference to specific embodiments thereof which are illustrated in the appended drawings. It is appreciated that these drawings depict only typical embodiments of the invention and are therefore not to be considered limiting of its scope. The invention will be described and explained with additional specificity and detail through the use of the accompanying drawings in which:

FIG. 1 is a perspective view of an exemplary electrosurgical instrument according to an embodiment of the present invention coupled to a radio frequency generator and an aspirator;

FIG. 1A is a perspective view similar to that of FIG. 1, showing an alternative instrument configuration;

FIG. 1B is a perspective view similar to that of FIG. 1, showing another alternative instrument configuration;

FIG. 1C is a perspective view similar to that of FIG. 1, showing yet another alternative instrument configuration;

FIG. 1D is a perspective view similar to that of FIG. 1, showing yet another alternative instrument configuration;

FIG. 1E is a perspective view similar to that of FIG. 1, showing an alternative instrument configuration, with one or more of the control buttons on the bottom surface of the handle portion of the instrument;

FIG. 2 is a close up view of the distal electrode end of the exemplary electrosurgical instrument of FIG. 1;

FIG. 3A shows a cross-sectional view through the distal end of the exemplary electrosurgical instrument of FIG. 1;

FIG. 3B shows a cross-sectional view through the distal end of another exemplary electrosurgical instrument;

FIG. 4A shows a cross-sectional schematic view of an exemplary spring loaded button for providing the boosted ablation mode;

FIG. 4B shows a cross-sectional schematic view of the spring loaded button of FIG. 4A, but with the button depressed, so as to provide boosted ablation;

FIGS. 4C-4D show perspective views of an exemplary button and the surrounding handle where the button is configured as a roller for providing the boosted ablation mode;

FIG. 4E shows a cross-sectional schematic view of the exemplary roller button of FIG. 4C-4D;

FIG. 4F shows a cross-sectional schematic view of the roller button of FIG. 4C-4D, but with the roller advanced, so as to provide boosted ablation;

FIG. 5 illustrates an exemplary operating room environment where arthroscopic surgery is being conducted on a knee of a patient, showing how the practitioner guides and manipulates an endoscopic instrument with one hand, and an electrosurgical instrument such as that of FIG. 1 with the other hand, simultaneously;

FIG. 6A shows a close up schematic view of the distal electrode end of an exemplary electrosurgical instrument, positioned adjacent tissue to be ablated, with active cooling of the electrode;

FIG. 6B shows a close up schematic view similar to that of FIG. 6A, but where active cooling of the electrode has been temporarily decreased or halted, providing a boosted ablation mode with a larger and/or more intense ablative sparking energy field provided by the electrode;

FIG. 7A shows a schematic view of the distal electrode end of an exemplary electrosurgical instrument, schematically illustrating the energy field generated during operation in the normal ablation mode, with active cooling of the electrode; and

FIG. 7B shows a view similar to that of FIG. 7A, illustrating how temporarily decreasing or halting cooling of the electrode causes the energy field to become enlarged.

### DETAILED DESCRIPTION

### I. Introduction

The present disclosure is directed to electrosurgical instruments for selectively operating in normal and boosted ablation modes in ablative capability (e.g., by providing increased sparking density at the electrode) without requiring an increase in current density at the electrode and related methods of use. For example, such an instrument may include an elongate probe having a handle portion and a distal end, at least one electrode disposed at a distal end of the elongate probe configured to perform ablation, and an aspiration lumen disposed within the elongate probe that opens at the distal end of the elongate probe so as to actively aspirate fluid adjacent the distal end of the elongate probe through the aspiration lumen opening and into the aspiration lumen while aspiration is applied to the aspiration lumen.

The instrument includes one or more user operable control components (e.g., buttons), disposed on the handle portion of the elongate probe. At least one such control component is configured to selectively restrict aspiration to the aspiration lumen so as to temporarily decrease or stop active suctioning of fluid, tissue debris and/or vapor bubbles through the aspiration lumen opening. Such functionality advantageously provides an ablative sparking energy field provided by the at least one electrode that is more dense and/or larger and/or more intense as compared to the sparking energy field generated while normal aspiration is applied. A boost in the rate of tissue ablation may advantageously be achieved without increases the current density at the at least one electrode.

For example, during normal operation, where the button or other control component which restricts aspiration is not activated, active cooling of the electrode occurs as irrigating saline or similar liquid is delivered to the site (e.g., from an adjacent separate instrument, such as an endoscope) and actively aspirated through a lumen adjacent to the electrode. This irrigating saline, along with tissue debris, bubbles and/or other materials resulting from the procedure, are suctioned into the aspiration lumen, which may open through the electrode. As a result, under such operation, saline continuously passes by the electrode surfaces, providing active cooling of the electrode and adjacent fluids.

Upon activation of the button or other control component to provide a boost to ablative capability, active cooling is temporarily decreased or suspended as aspiration of fluid is slowed or stopped. This causes any saline and other materials adjacent the electrode to quickly be heated. Because additional saline is not being actively drawn toward the electrode surfaces, materials in the immediate vicinity of the electrode are more quickly vaporized or ablated. Increasing the quantity of water vapor near the electrode surface increases electrical resistance, which causes even higher sparking density when operating the electrode at the same current density as in normal ablation mode. As a result, a more dense and/or larger and/or more intense ablative sparking energy field is generated, and tissue adjacent the electrode is much more quickly and effectively ablated. Even if active aspiration is not employed (e.g., by wall suction or peristaltic pump) and passive outflow is allowed, because of a positive pressure difference between the fluid at the electrode end of the lumen, some fluid flow may still occur (e.g., unless the lumen is completely blocked). For example, there may be about 30 to about 80, or about 50 to about 80 mm Hg of a pressure differential between the electrode end of the aspiration lumen and the proximal end of the aspiration lumen.

It has been observed that the distal tip with the electrode can light up like a flamethrower or blow torch almost immediately after active cooling is slowed or stopped. This allows the practitioner to cut through or ablate tissue which proved to be more difficult under the previous otherwise similar conditions where fluid aspiration actively cools the electrode. Such increased ablative capability is made possible even without providing an increase in electrical power and/or current density to the electrode and/or without decreasing the electrode surface area. The practitioner may thus cut through, ablate and remove more difficult portions of tissue by actuating such a button or other control component. Following a burst of increased tissue ablation ability, deactivation (e.g., release) of the button may cause or permit fluid aspiration to resume, which clears away debris in the stream of irrigant drawn into the aspiration lumen opening and actively cools the electrode.

### II. Exemplary Electrosurgical Instruments and Related Methods

Following are exemplary configurations that can be used in or as part of the inventive apparatus and methods. Notwithstanding the following descriptions relative to how the illustrate user-operated control components may function in the described embodiments, it should be understood that the illustrated user-operable buttons or other control components can be modified to provide other functionalities as desired. For example, one, some, or all of the illustrated buttons and foot pedals (or other control components known in the art) can be configured to function as a toggle switch, such as activating a specified function when placed in a first position (or actuated a first time) and deactivating the function and/or providing a different function when placed in a second position (or actuated a second time). Alternatively, one, some, or all of the illustrated buttons and/or foot pedals can be configured so as to only activate a specified function when continuously actuated by the user, such as a safety switch or button that only activates an electrode or other function, such as reducing or cutting off aspiration, when depressed.

In some embodiments, aspiration can be initiated or stopped independently of one or more user-operated controls that operate the electrode features. In other words, the device can be continuously aspirating or not aspirating independently of how or when a user manipulates one or more controls to operate the electrode features. In other embodiments, aspiration can be initiated, stopped, or reduced using one, some, or all of the one or more user-operated controls that operate the electrode features.

In embodiments where continuous aspiration is provided independently of the user-operated control components that operate the electrode features, a first user-operated control component can be actuated to deliver power to an ablation electrode and place the apparatus in normal ablation mode. A second user-operated control component can be actuated to deliver power to a coagulation electrode and place the apparatus in coagulation mode. A third user-operated control component can be actuated to place the apparatus in boosted ablation mode, with power being delivered to the ablation electrode while simultaneously reducing or cutting off aspiration. In some embodiments, at least the third user-operated control component can be a safety switch that only causes boosted ablation when continuously actuated by the user. Release of the third control component can automatically cease boosted ablation, e.g., by restoring aspiration and/or cutting off power to the ablation electrode. In some embodiments, the first and third user-operated controls can be safety switches so that the first user-operated control must be continuously actuated to place the apparatus in normal ablation mode and both the first and third user-operated controls must be continuously actuated to place the apparatus in boosted ablation mode. Depending on the device design and/or user selection, releasing or deactivating the third control to switch out of boosted ablation made may switch the apparatus back to normal ablation mode, switch the apparatus to coagulation mode, or cease all electrode function, with continued aspiration or cessation of aspiration.

In other embodiments, aspiration may be initiated, stopped, or reduced using one, some, or all of the one or more user-operated control components that operate the electrode features. In other words, the device may only begin or cease aspiration when a user manipulates one or more controls to operate the electrode features. By way of example, a first user-operated control component can be actuated to initiate aspiration and also deliver power to an ablation electrode to place the apparatus in normal ablation mode. A second user-operated control component can be actuated to initiate aspiration and also deliver power to a coagulation electrode to place the apparatus in coagulation mode. A third user-operated control component can place the apparatus in boosted ablation mode by delivering power to the ablation electrode and not initiating aspiration or only initiating partial aspiration. As above, at least the third user-operated control component can be a safety switch that only causes boosted ablation while being continuously actuated by the user. Release of the third control component can automatically stop boosted ablation, e.g., by cutting off power to the ablation electrode and/or restoring aspiration if the first user-operated control component has been actuated (if a toggle switch) or is being continuously actuated (if a safety switch). Depending on the device design and/or user selections, switching the apparatus out of boosted ablation mode may switch it to normal ablation mode, switch it to coagulation mode, or cease all electrode function.

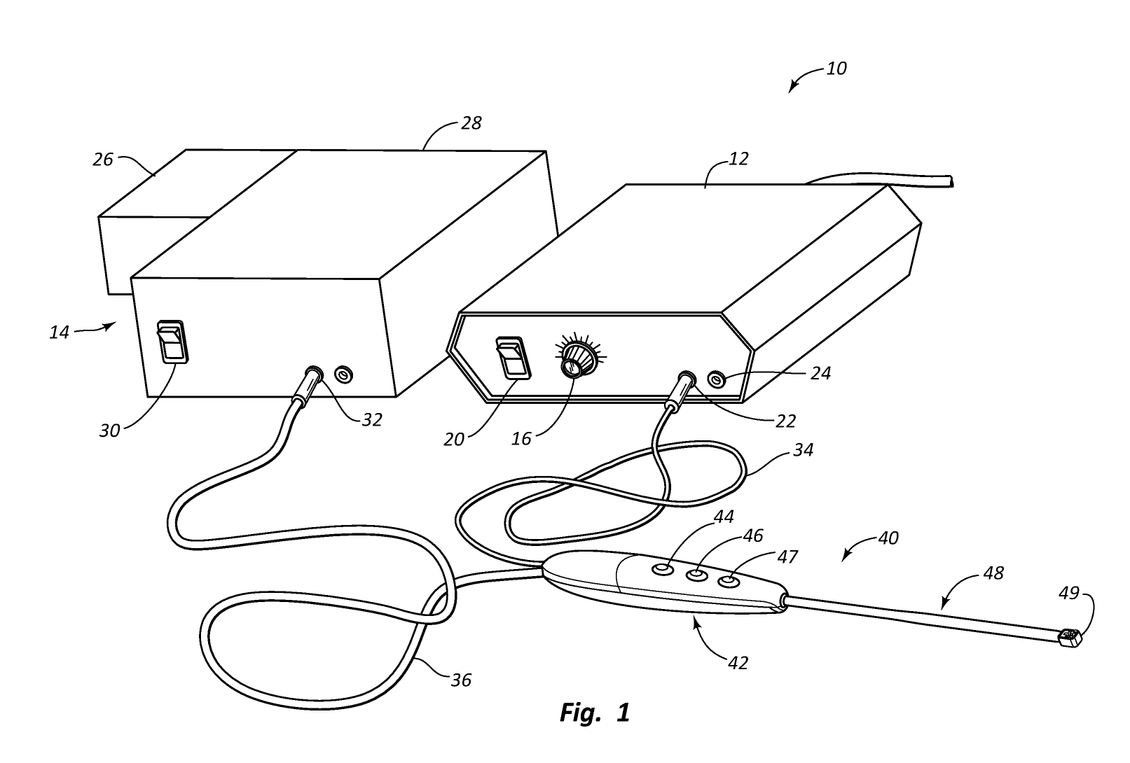

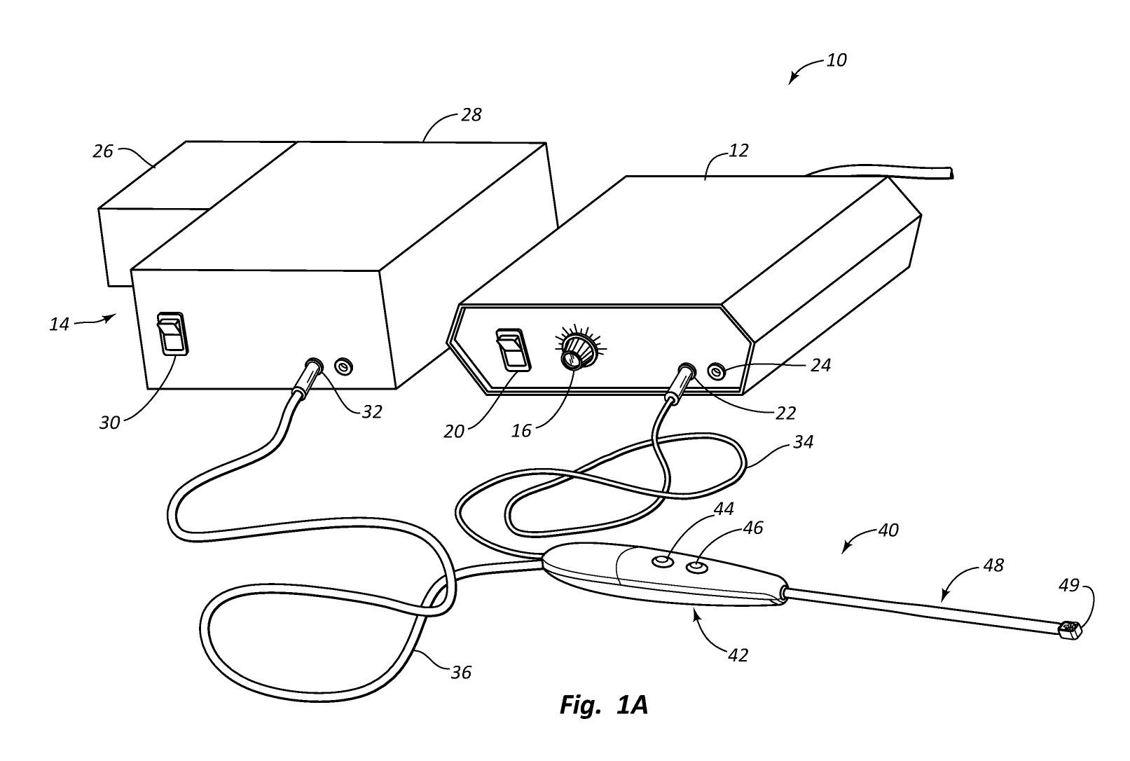

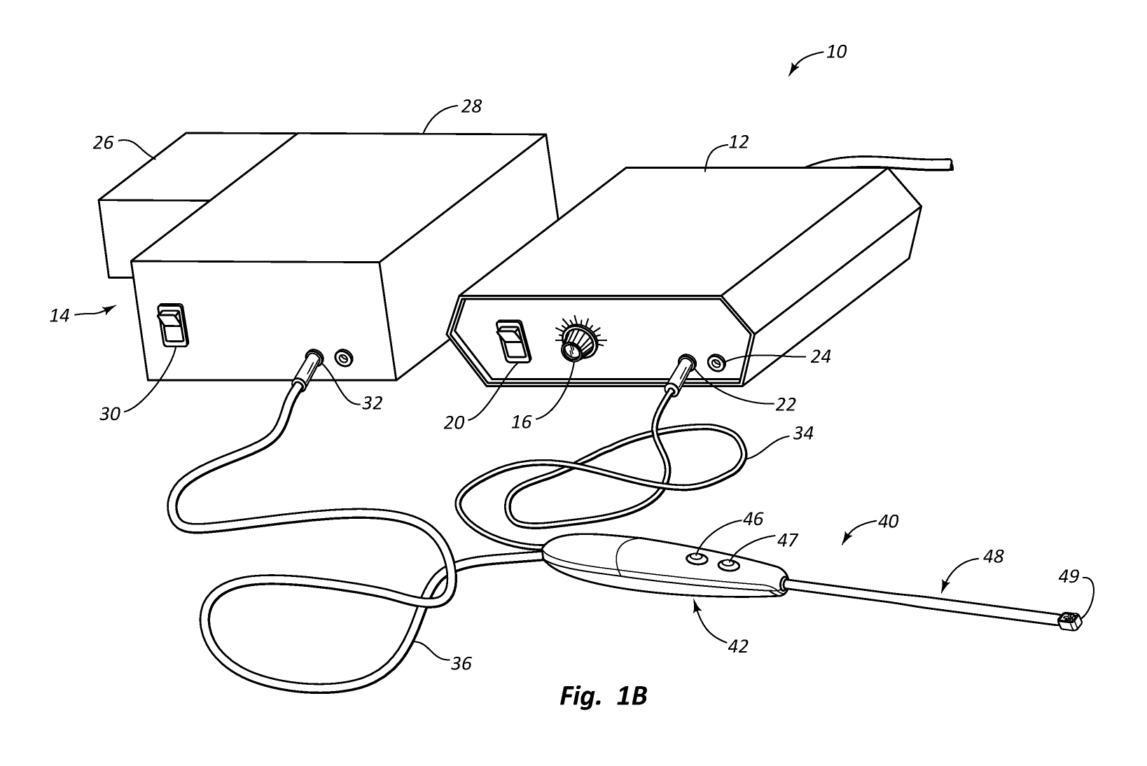

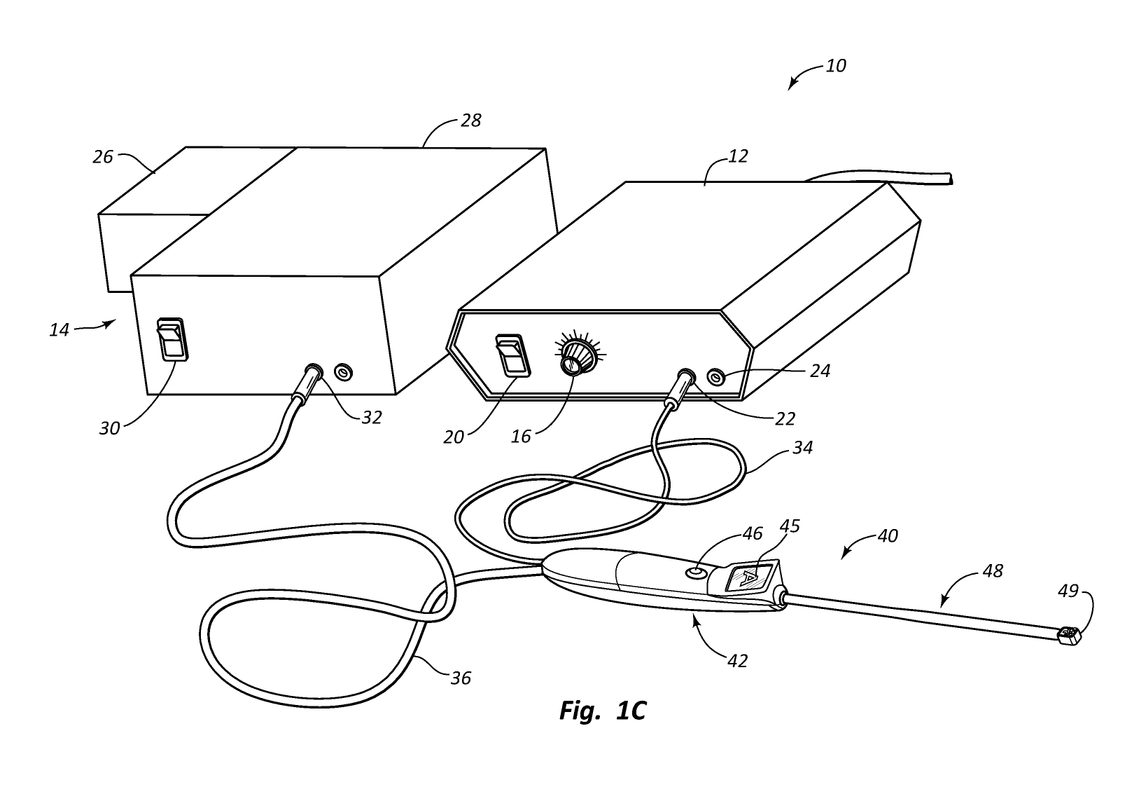

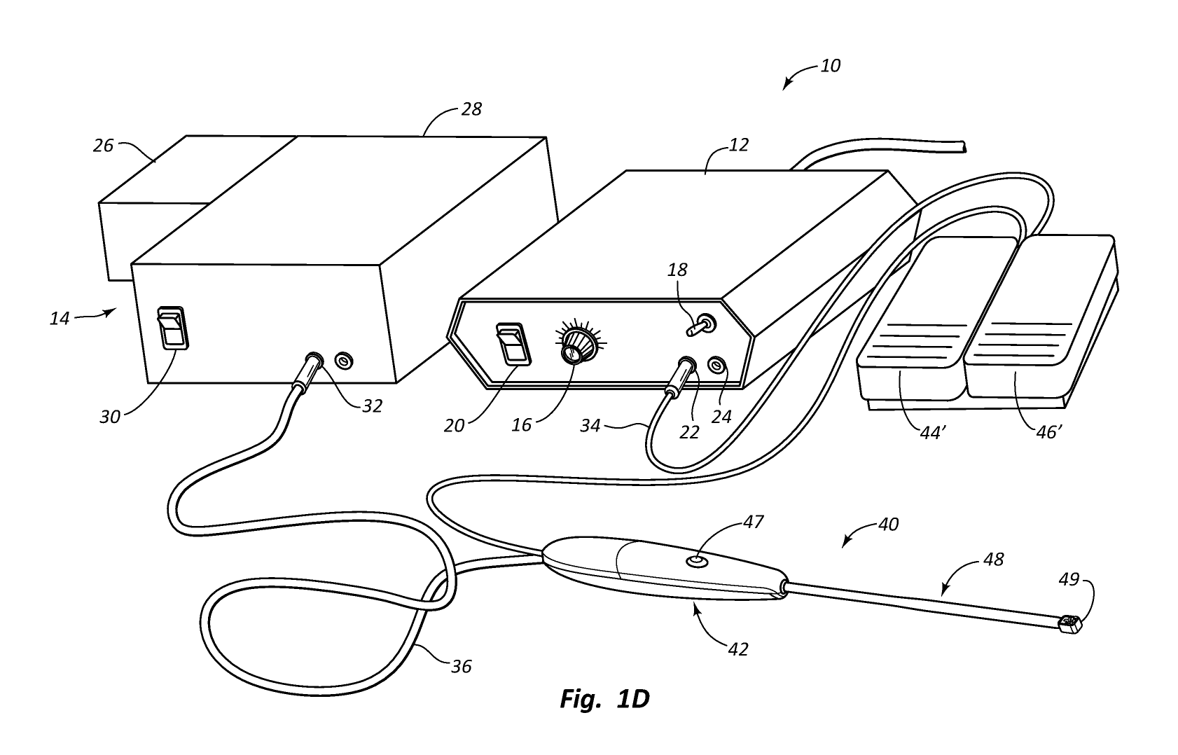

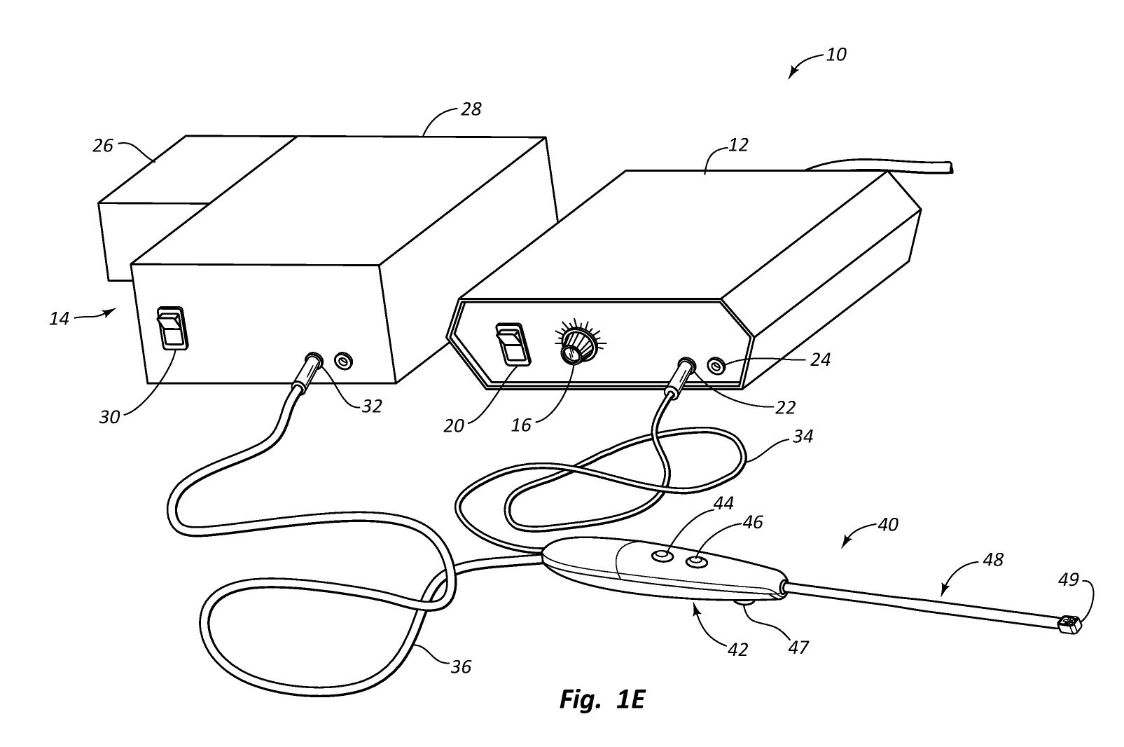

FIG. 1 illustrates an exemplary electrosurgical system according to some embodiments of the invention. The electrosurgical system 10 includes an electrosurgical instrument 40 that is electrically coupled to an electrosurgical generator 12 and an aspirator 14. Aspirator 14 can be configured to provide or cease aspiration independently of user-operated switches or other control components for controlling electrode function. Alternatively, aspirator 14 can be configured to provide or cease aspiration in conjunction with user-operated switches or other control components for controlling electrode function.

Electrosurgical generator 12 may be configured to generate radio frequency (“RF”) wave forms. Generator 12 can generate power useful for ablating tissue and optionally coagulating tissue. In one embodiment, generator 12 may include standard components, such as dial 16 for controlling the frequency and/or amplitude of the RF energy, a switch 20 for turning the generator on and off, and an electrical port 22 for connecting the electrosurgical instrument 40. Generator 12 may also include a port 24 for connecting an electrical ground or a return electrode. It will be appreciated that generator 12 can be designed for use with both bipolar and monopolar electrosurgical instruments. Bipolar instruments include a return electrode on the electrosurgical instrument itself (e.g., at the distal end thereof). Monopolar instruments may not include a return electrode, as the return electrode may be provided separately. Generator 12 may be designed to operate at constant electrical current through the electrode and/or at constant power in order to avoid unwanted bursts of electrical current and/or power through the patient.

Aspirator 14 may include a pump 26, a reservoir 28, an on/off switch 30, and an aspirator port 32. Pump 26 provides negative pressure for aspirating fluids, gasses, and debris through electrosurgical instrument 40. Aspirated fluids and debris can be temporarily stored in reservoir 28. In another embodiment, electrosurgical instrument 40 may be connected to wall suction. When using wall suction, canisters or other reservoirs may be placed in the suction line to collect aspirated debris and fluids. Those skilled in the art will recognize that many different configurations of generator 12 and aspirator 14 can be used in the present invention.

Electrosurgical instrument 40 is depicted as an elongate probe and includes a power cord 34 for providing electrical power to instrument 40 from generator 12 through electrical port 22. Extension tubing 36 may provide a fluid connection between instrument 40 and aspirator 14. Electrosurgical instrument 40 may include a handle portion 42 and a distal end portion 48. In one embodiment, handle portion 42 provides an enlarged, easily grippable handle for instrument 40. Distal end portion 48 of instrument 40 may include an electrode head 49, which includes one or more electrodes, as well as an opening for an aspiration lumen.

Instrument 40 may be configured to only ablate tissue or, alternatively, so as to selectively coagulate or ablate tissue. Handle portion 42 is shown as including three buttons 44, 46, and 47, or other control components that may be easily operated by the practitioner, without requiring the practitioner to release his or her grip on handle portion 42. Thus, buttons 44, 46, and 47 may be easily and conveniently manipulated during a surgical procedure (e.g., by reaching and pressing with a thumb of the gripping hand). Two buttons (e.g., 44 and 46) may allow the practitioner to select or switch between coagulation mode (e.g., button 44) and ablation mode (e.g., button 46). Button 47 may activate the boosted ablation mode by temporarily restricting aspiration of fluids through the lumen and active cooling of electrode head 49.

Providing controls for selecting between coagulation mode, ablation mode, and boosted ablation mode on proximal handle portion 42 is advantageous, as during a typical arthroscopic or similar procedure, the practitioner grips and manipulates an instrument such as 40 in one hand, and another instrument (e.g., an endoscope) in the other hand (e.g., see FIG. 5). Thus, with both hands occupied, it can otherwise be difficult and impractical to manipulate controls that are disposed elsewhere (e.g., on generator 12, aspirator 14, etc.). Placement of the controls on handle portion 42 is particularly beneficial as this provides the practitioner with greater flexibility in the mode of operation, and specific operational characteristics provided by instrument 40, without requiring help from an assistant, release of a hand from handle portion 42, etc.

In an embodiment, as illustrated, the button or other control component 47 for boosting ablative capability may be disposed adjacent to the control component 46 for selecting the ablation mode (e.g., and not adjacent an optional control component 44 for selecting a coagulation mode, where a coagulation mode is provided). Such placement may be beneficial, as the practitioner may select the ablation mode by pressing button 46 (e.g., with the thumb), and if insufficient ablative capability is being provided, the practitioner may slide the thumb upwards to button 47, depressing button 47 so as to temporarily deliver increased ablative capability. Because button 47 is in sufficiently close proximity to button 46 (e.g., no buttons disposed therebetween), this can be accomplished using the thumb, without the practitioner having to release his or her grip of the proximal handle portion 42.

One, some, or all of buttons 44, 46, and 47 may be toggle switches, safety switches, or combinations of toggle and safety switches. In some embodiments, only button 47 is a safety switch and buttons 44 and 46 are toggle switches such that boosted ablation only occurs while button 47 is continuously actuated (e.g., depressed). In other embodiments, button 46 is also a safety switch such that normal ablation only occurs while button 46 is actuated (e.g., depressed). In some cases, actuating button 47 results in boosted ablation. In other cases, buttons 46 and 47 must be actuated simultaneously to provide boosted ablation.

In some embodiments, such as where buttons 46 and 47 are both safety switches, the practitioner may be required to continuously depress button 46 for normal ablation and both buttons 46, 47 simultaneously for boosted ablation. In another embodiment, where button 46 is a toggle switch and has already been actuated, actuating button 47 will permit the instrument to continue operating in ablation mode while also cutting off or restricting aspiration to provide boosted ablation. A configuration requiring simultaneous depression of buttons 46, 47 for boosted ablation may be preferred for safety reasons (e.g., upon release of any given button, the functionality previously provided by depression of that button ceases).

In some embodiments, one or more of the control buttons may be disposed on another surface of the instrument 40. For example, buttons 44, 46, and 47 are shown in FIG. 1 as being disposed on a top surface of handle portion 42, although in another embodiment, one or more of buttons 44, 46, or 47 could be disposed on a bottom surface of handle portion 42. For example, button 47 could be disposed on the bottom side of handle portion 42, allowing the practitioner to depress button 46 (or 44) with the thumb on the top of handle portion 42, while button 47 could be depressed with a finger of the same hand. This may be particularly beneficial where the instrument may require simultaneous pressing of the ablation button (e.g., 46) and the ablation boost button (e.g., 47) to operate in boosted ablation mode. Such an embodiment is shown in FIG. 1E.

In another embodiment, only two buttons (e.g., 44 and 46) may be provided, where button 46 may serve both to select normal ablation when actuated a first time and initiate boosted ablation when actuated a second time. FIG. 1A illustrates such an embodiment. For example, upon first pressing button 46, a “normal” ablation mode may be selected. In order to provide increased ablative capability, the practitioner may press button 46 again. In an embodiment, button 46 may be held down so long as the increase in ablative capability is desired. Release of button 46 (or pressing it a third time) may either restore the device to normal ablation mode by resuming fluid aspiration and active cooling of the electrode, or it may cease ablation entirely until actuated again. The control components could also be configured to cancel the boosted ablation mode by pressing one of the other buttons (e.g., button 44).

While the user operable control components are illustrated as buttons 44, 46, and 47, it will be appreciated that any suitable user operable control components, including but not limited to buttons, switches, a touch screen, etc. may be suitable for use. The user may select between two basic operational modes with control components 44 and 46, and may select a boosted ablation mode by actuating control component 47 (e.g., when in the ablation mode). The control components 44 and 46 can be any type of mechanical or electrical input device which upon actuation causes the desired change in delivery of electrical power, and/or a change in the amount of active electrode surface area.

In an embodiment, the control component 47 for providing boosted ablation may be configured to simultaneously begin delivery of power to the ablation electrode 50 and cut off or reduce aspiration. Thus the electrode may be activated independently and simultaneously by button 47 (while button 46 may independently provide for activation of electrode 50 for the normal ablation mode). In such an embodiment, when button 47 is no longer actuated, not only may aspiration be restored, but electrical power to the electrode may also be cut off.

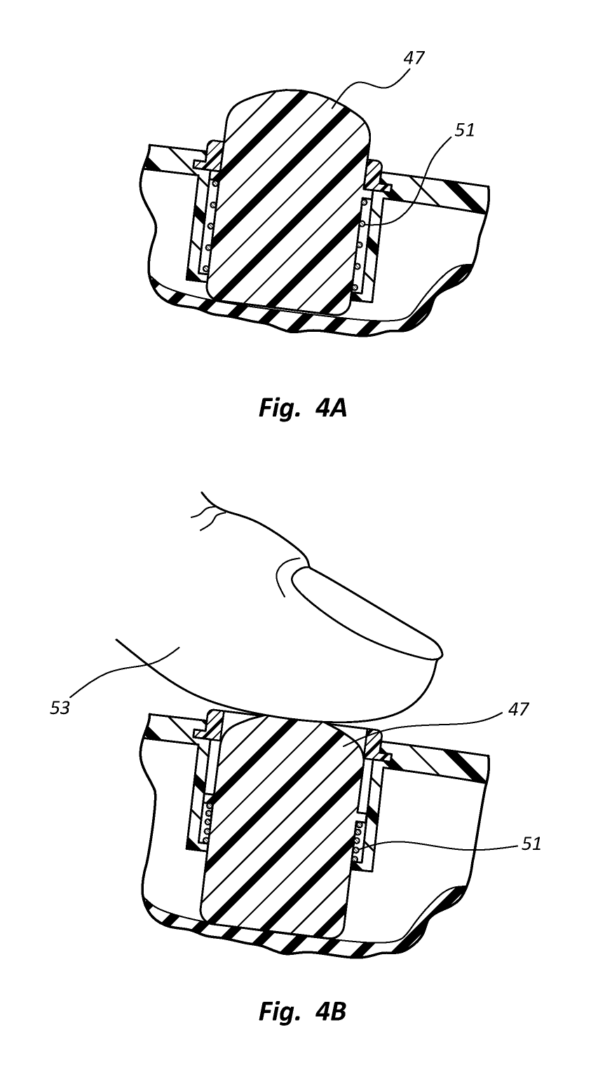

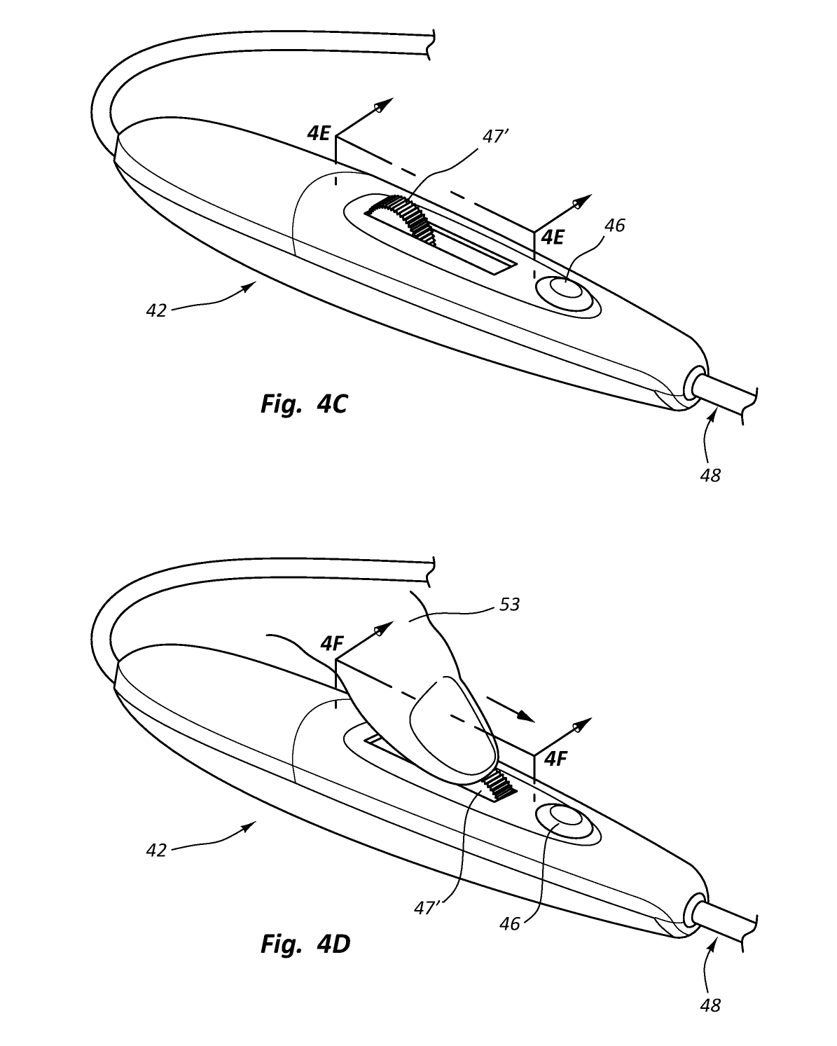

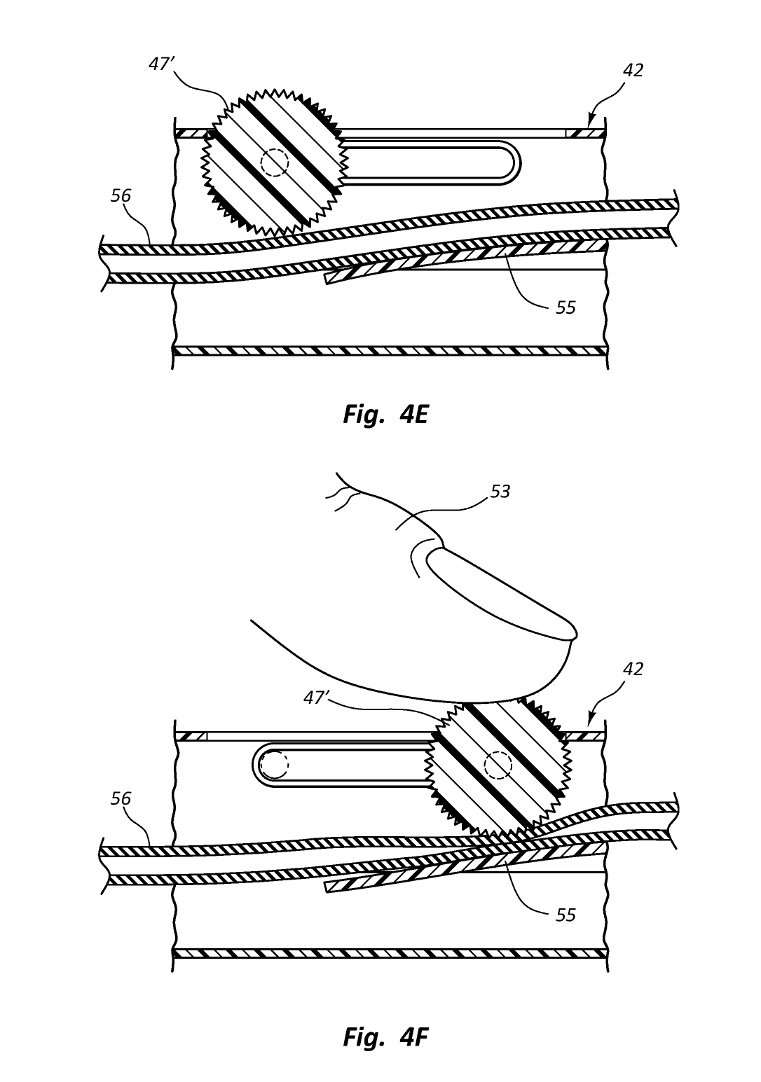

For safety reasons, the preferred configuration for at least button 47 may be a spring loaded safety button or otherwise default to an unactivated or deactivated condition, so that power is only delivered to the electrode when the button is actively depressed or otherwise continuously actuated. FIGS. 4A-4B, described in further detail below, show an exemplary spring activated button 47. It will be appreciated that any of the buttons or other control components may be spring loaded or similarly configured to shut off when not actively depressed (or actuated). For example, depression of spring loaded button 47 may complete an electrical circuit providing electrical power to the electrode. Depression of spring loaded button 47 may also cut off or at least reduce aspiration.

Control component 47 may similarly be any type of mechanical, electrical, or other input device which upon actuation selectively decreases or cuts off aspiration to aspiration lumen opening 52 so as to slow or suspend fluid aspiration and active cooling of electrode 50 so long as control component 47 is actuated. For example, activation of button 47 may send an electrical or other signal to aspirator 14 instructing it to decrease or cut off aspiration. In another embodiment, activation of button 47 may mechanically occlude or close off aspiration lumen 56 (e.g., through a roller, a valve, etc.), preventing suction from being applied over opening 52. In any case, when active cooling is reduced or eliminated, electrode 50 provides significantly greater ablative capability manifest as an ablative sparking energy field that is larger and/or more intense while so actuated. For example, it has been observed that the electrode distal end of the instrument nearly immediately lights up like a flamethrower or blow torch, so long as such active cooling is suspended. Upon release of control component 47 or other cancelling of the boosted ablation mode, fluid flow and active cooling are restored, returning the device to normal ablation mode.

It will be appreciated that a device which does not include a coagulation mode may be employed, e.g., including a button 46 to activate ablation, and another button 47 to enter a boosted ablation mode (FIG. 1B). Similarly, as described above, it will be apparent that a single button may control both the ability to enter the normal ablation mode (where fluid flow and active cooling are provided) and a boosted ablation mode (where fluid flow and associated cooling are temporarily decreased or suspended). Such a button may also provide for a coagulation mode, if desired, such as by reducing power to the electrode. For example, such a single button embodiment is shown in FIG. 1C, otherwise similar to FIG. 1, but without buttons 44 and 47. Upon first pressing button 46, the ablation mode may be activated. In order to provide increased ablative capability, the practitioner may press button 46 again. In an embodiment, button 46 may be held down so long as the increase in ablative capability is desired. Release of button 46 (or pressing it a third time) may resume fluid flow, active cooling, and normal ablation by the electrode.

Where the single button is configured to provide coagulation as well, pressing it (e.g., button 46 of FIG. 1C) a first time may select coagulation, pressing it a second time may select ablation, and pressing it a third time may select boosted ablation. A display 45 may also be provided to provide an indicator of which mode is currently selected, e.g., displaying C for coagulation, A for ablation and BA or some other indicator designating the boosted ablation mode. It will be apparent that any indicator scheme may be employed, and that such a display may be included with any of the other device configurations disclosed herein.

In another alternative, one or more control components (e.g., a button) may be configured to switch into an ablation mode from a coagulation mode by decreasing or cutting off aspiration. For example, in a coagulation mode, upon restricting or cutting off active suction of cooling saline, the energy field generated by the electrode may then be sufficiently intense to provide ablation, rather than coagulation. Thus, a user may operate the device in coagulation mode and, by pressing the button which decreases or cuts off aspiration and associated cooling, may enter an ablation mode without increasing the provided electrical power.

It is not necessary that the controls for switching between a coagulation mode and the normal ablation mode be configured as button(s) positioned on the handle of the instrument. For example, in another embodiment, a foot pedal may be provided which may allow selection of the coagulation mode or the ablation mode. Such an embodiment may include a single button or other user operable control component 47 disposed on the handle portion for restricting aspiration, and providing a boosted mode of operation. As described herein, such a boost may be selected and provided whether in a coagulation mode or an ablation mode, in any of the embodiments described herein. FIG. 1D illustrates a system as described above including a single button 47 (e.g., similar to FIG. 1C), but also including foot pedals 44′ and 46′ for selecting a coagulation mode (e.g., pedal 44′) or a normal ablation mode (e.g., pedal 46′). In another embodiment, selection of the coagulation or normal ablation mode could be achieved with a switch 18 or similar control component (e.g., on generator 12). Of course, such a switch may be less preferred, as it is not readily accessible to the practitioner without a “third” hand.

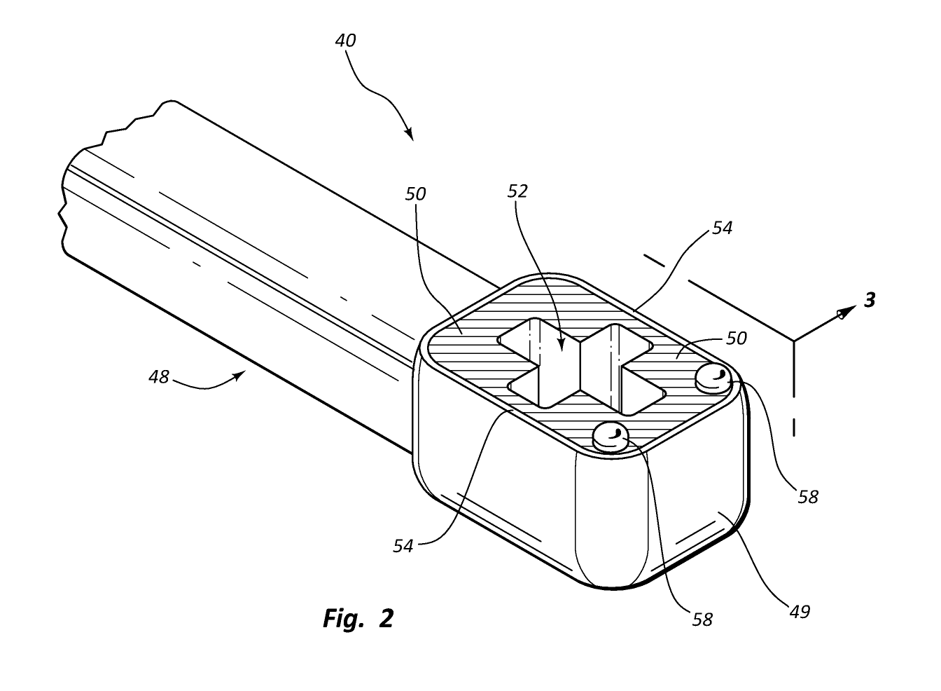

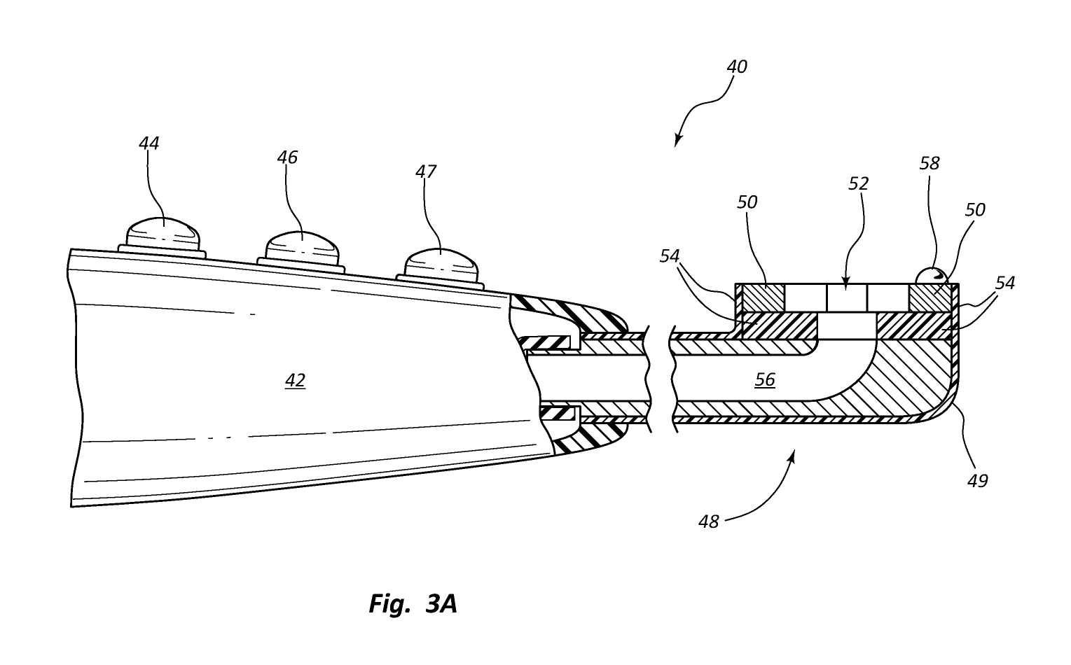

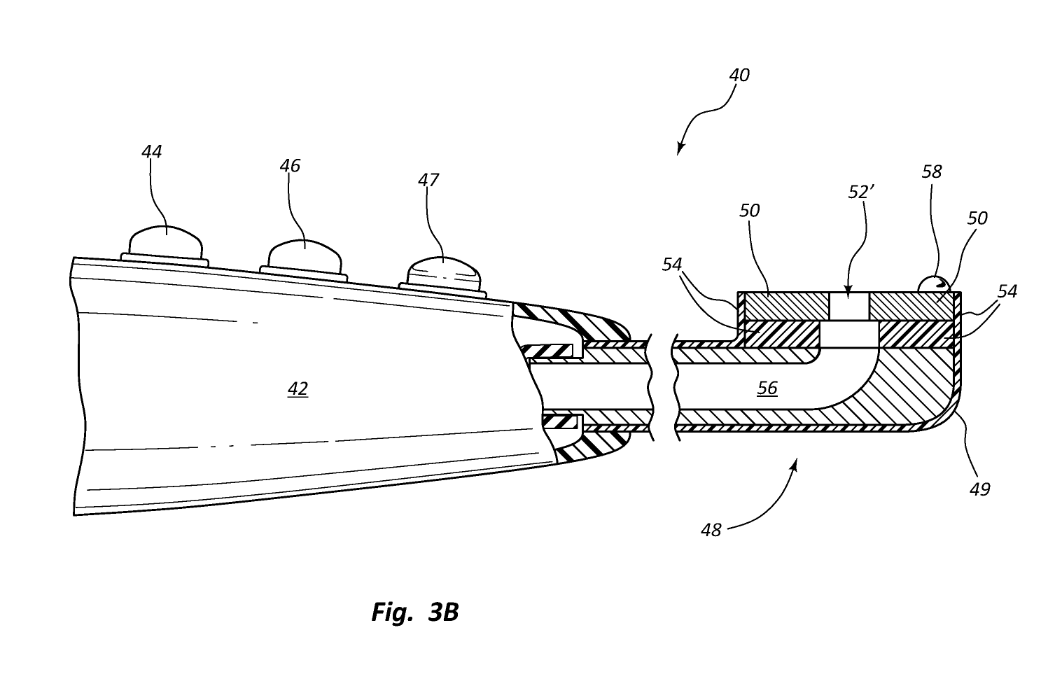

FIGS. 2 and 3A illustrate a close up view and cross-sectional view, respectively of an exemplary embodiment of an electrode configuration. As illustrated, instrument 40 may include an electrode 50 on distal end portion 48, which electrode 50 is exposed so as to allow its contact with tissue to be coagulated or ablated. Electrode 50 may be a conductive element such as a metal or other suitable material for conducting an electrical current. Electrode 50 may be electrically insulated from the remainder of instrument 40 by insulating material 54 (e.g., a ceramic). Electrical power may be delivered to electrode 50 from generator 12 and cord 34 through appropriate electrical traces or other wiring (not shown).

As seen in FIGS. 2-3A, in an embodiment, electrode 50 may be configured so as to include one or more sharp angled edges (e.g., as opposed to smoothly curved edges), e.g., adjacent opening 52 of aspiration lumen 56. In an embodiment, the opening 52 of aspiration lumen 56 may be disposed through electrode 50, and may be other than circular, oval, or other rounded shape. For example, the opening may include a cross-section that is polygonal in shape, so as to define one or more sharp edges in adjacent electrode 50, as perhaps best seen in FIG. 2. For example, FIG. 2 shows a cross-shaped geometry for opening 52 of aspiration lumen 56. One or more bumps or protrusions 58 extending upwardly from electrode surface 50 may be provided, as seen in FIG. 2. FIG. 3A shows a cross-sectional schematic view through a portion of instrument 40, illustrating protrusions 58, as well as opening 52 of aspiration lumen 56. Aspiration lumen 56 may extend within instrument 40, with opening 52 being positioned within electrode 50. Aspiration lumen 56 can be used with aspirator 14 (FIG. 1) to withdraw debris and fluids from the surgical site during ablation and/or coagulation.

Electrode 50 may be configured to provide ablation when instrument 40 is in the ablation mode. Electrodes configured for ablation may have a relatively small surface area, so that the power provided by generator 12 to electrode 50 is sufficient to create a plasma in the aqueous medium. In an embodiment instrument 40 may include a power output of from about 150 W to 400 W, more preferably about 200 W to 400 W. Applicable regulatory requirements within the U.S. limit power delivery of such electrosurgical instruments to no more than 400 W. For a power rating of 400 W, the active surface area can be in a range from about 3 mm² to about 30 mm², more preferably about 5 mm² to about 25 mm², and most preferably about 7 mm² to about 20 mm².

Although FIG. 2 illustrates a single active electrode, it will be appreciated that more than one electrode may be provided (e.g., electrically isolated from one another). For example, a separate electrode may be provided, which may or may not be operated in combination with electrode 50 for increased electrode area when in a coagulation mode. In addition, in a bipolar instrument, a return electrode may be provided on distal end 48. By way of example, the inventor's U.S. Pat. No. 8,394,088, discloses further details of such systems. The above referenced patent is herein incorporated by reference in its entirety.

Electrode 50 is shown as providing a continuous surface area. In an alternative configuration, the one or more electrodes may comprise a plurality of distinct surface areas each separated by an insulating material. An example of such an electrode is shown in U.S. Pat. No. 8,394,088, incorporated by reference above.

Instrument 40 may switch between ablation and coagulation modes by changing the amount of power provided to electrode 50, by activating an additional electrode to increase surface area for a coagulation mode (e.g., at the same power), or both. For example, when switching from an ablation mode to a coagulation mode, the power provided to the electrode(s) may be decreased, and/or the surface area of active electrode(s) may be increased. In any case, such selection results in a decrease in power density per electrode surface area. When selecting the ablation mode, the power density per electrode surface area is sufficiently high to form a plasma, while in the coagulation mode, the power density per electrode surface area is lower, and may not result in plasma formation, but may be sufficient to coagulate tissue adjacent the electrode(s). Of course, in some embodiments, the instrument may not provide a coagulation mode.

When in the ablation mode and selecting the boosted ablation mode (so as to move from one to the other), no increase in delivered electrical power may be associated with the change. For example, a given amount of power up to 400 Watts may be provided to the electrode when in the ablation mode, and the same amount of electrical power may be delivered when switching to the boosted ablation mode. Even so, as described herein, a more dense ablative sparking energy field is provided. In an embodiment, the ablative sparking energy field may increase in density by at least about 10%, at least about 20%, at least about 35%, at least about 50%, at least about 75%, at least about 100%, at least about 150%, or at least about 200%.

FIG. 3B illustrates a configuration similar to that of FIG. 3A, but in which the opening 52′ into lumen 56 is smaller than the underlying dimension of lumen 56. Such an embodiment may aid in preventing plugging of lumen 56, as if a piece of debris is sufficiently large to pass through opening 52′, it will easily pass through lumen 56 to storage reservoir 28. For example, opening 52′ may have a width or diameter dimension (for circular openings) that is smaller than the width or diameter of lumen 56, adjacent opening 52′. Of course, other embodiments are also possible, where the width or diameter of the opening is greater than the width or diameter of the lumen at a location adjacent the opening (e.g., FIG. 3A shows such an embodiment).

FIGS. 4A and 4B illustrate schematic views of button 47, which may be spring loaded with spring 51 so as to default or be biased to an unselected configuration. FIG. 4A shows button 47 in the default, unselected configuration. FIG. 4B shows button 47 in the depressed configuration (e.g., with thumb 53). The practitioner may depress and hold down button 47 so long as the boosted ablation mode is desired. While depressed, aspiration to opening 52 may be temporarily decreased or cut off to temporarily slow or suspend the flow of cooling irrigant fluid adjacent to electrode 50, providing the desired increased ablative capability. Once the spring loaded button 47 is released, normal aspiration, fluid flow, and associated cooling of electrode 50 may resume.

In another embodiment, spring loaded button 47 may include 2-stage function, by which button 47 locks in a depressed condition once pressed, and by which the button can be released by pressing it again. As described above, the spring loaded button cuts off aspiration and provides the ablative sparking energy field of increased size or intensity when in the depressed condition, normal aspiration being restored once the spring loaded button is pressed again, releasing the spring loaded button. Such a configuration provides an advantage in that the practitioner is not required to hold the button in a depressed condition for the desired duration of boosted ablation, but may simply depress the button, which locks in that depressed condition. Once boosted ablation is no longer desired, the practitioner simply presses the depressed button again, unlocking it so that it returns to its undepressed condition (FIG. 4A).

FIGS. 4C-4F illustrate another button embodiment, where button 47′ is configured as a roller that may mechanically occlude or close off (e.g., pinch) aspiration lumen 56, as roller 47′ is advanced. As seen in FIGS. 4C and 4D, roller button 47′ may be disposed within handle portion 42 rather than elsewhere within system 10, so as to be easily accessible to the practitioner's thumb 53. FIG. 4E illustrates roller button 47′ and lumen 56 before advancement, so that lumen 56 is not pinched closed, while FIG. 4F illustrates roller button 47′ having been advanced so as to at least partially occlude or pinch lumen 56, reducing or cutting off aspiration therethrough. As shown, lumen 56 may be positioned within handle portion 42 so as to include a ramped portion (e.g., supported by ramp support 55). In another embodiment, roller 47′ may ride down an incline as it is advanced, so as to impinge upon lumen 56 (e.g., where lumen 56 may extend straight through body 42).

Any number of other controls (e.g., buttons) may be provided on handle portion 42 with roller button 47′ for selecting a coagulation or normal ablation mode. For example, the illustrated embodiment shows a button 46 (e.g., which may select an appropriate mode). It will be appreciated that another button (e.g., button 44) may also be provided, or selection may be through foot pedal(s) or other controls, as described herein.

FIG. 5 illustrates an exemplary operating room environment where arthroscopic surgery may be performed on a knee of a patient, and illustrates how a practitioner may typically be required to grasp electrosurgical instrument 40 in one hand, while grasping an endoscope 60 in the other hand, as both instruments are inserted within the knee or other surgical site of the patient. The practitioner may thus be required to manipulate both instruments simultaneously, observing a video feed from the endoscope 60 on monitor 62. FIG. 5 illustrates a monopolar configuration, where a return electrode 63 having a relatively large surface area may be electrically connected to another portion of the patient (e.g., on a leg, etc.). Alternatively, a bipolar configuration may be employed where the probe of instrument 40 includes a return electrode on the instrument itself.

Saline or a similar irrigation liquid may be provided to endoscope 60 from bag 64 (e.g., through tubing 66). Thus, endoscope 60 may serve to provide irrigating fluid to the surgical site, which aids in capture and carrying away of debris generated in the procedure. Such irrigation fluid and debris is actively withdrawn from the surgical site through aspiration opening 52, allowing the practitioner to monitor the progress of the procedure on monitor 62. By way of example, when the practitioner actuates the boosted ablation mode by pressing or otherwise actuating control component 47, active suctioning of irrigation fluid and debris is temporarily decreased or halted, so as to provide the desired boost in ablative capability and rate of ablation. As a result of the reduction of active irrigation and continuous withdrawal of debris, the field of view shown on monitor 62 may become cloudy or hazy the longer the boosted ablative mode is maintained. As a result, in an embodiment, the practitioner may remain in the boosted ablative mode for only a short period of time (e.g., about 5 to about 10 seconds), may then resume aspiration so as to clear the field of view, and then may again select the boosted ablative mode (e.g., for about another 5 to about 10 seconds), if needed. The periods of boosted ablation mode and intervening clearing periods may be repeated as many times as needed. The clearing period (during which normal fluid aspiration is restored) between use of the boosted ablative mode periods may similarly last from about 5 to about 10 seconds, depending on how much clouding debris is to be cleared away. Such time periods may be as short as 1 second, or any interval above 1 second.

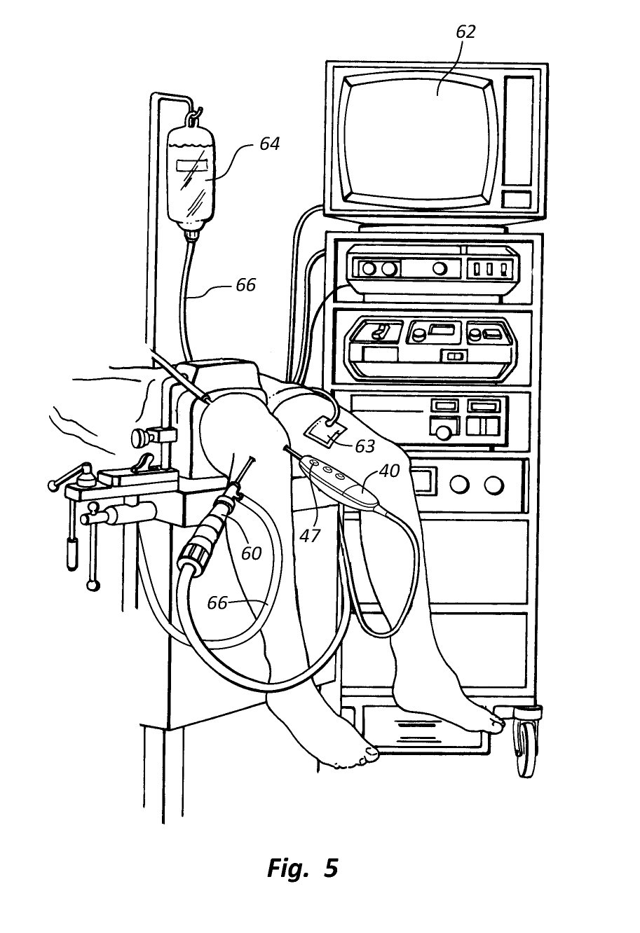

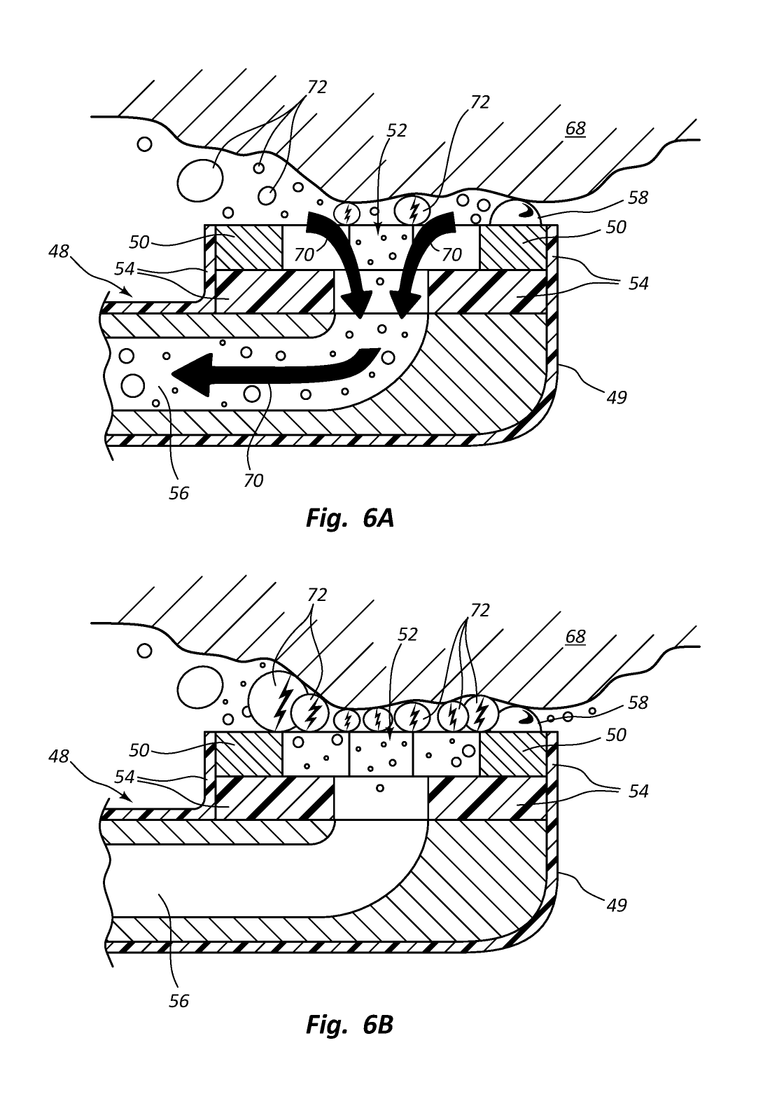

FIGS. 6A and 6B illustrate close up schematic views of the electrode distal end 48 and head 49 of instrument 40 as it is being used in a normal ablation mode (FIG. 6A), and in the boosted ablation mode (FIG. 6B). As seen in both FIGS. 6A and 6B, heating of electrode 50 causes formation of tiny bubbles 72 as the adjacent irrigating fluid is vaporized. Arcing occurs across some such formed bubbles between the surface of electrode 50 and adjacent tissue 68, resulting in formation of the desired plasma, which ablates a superficial depth of the adjacent tissue (e.g., about 50 μm to about 100 μm). As shown in FIG. 6A, irrigating fluid and debris carried therein are actively suctioned through opening 52 into aspiration lumen 56, represented by arrows 70. Such active suctioning of irrigating fluid near electrode surfaces 50 provides active cooling of electrode 50 and adjacent fluid.

FIG. 6B shows instrument 40 in a boosted ablation mode, with restricted aspiration of fluids and reduced fluidic cooling of electrode 50. Because of the temporary deliberate decrease in fluidic cooling, irrigating fluid and debris adjacent electrode 50 is quickly heated, resulting in generation of more water vapor and plasma, which decreases electrical conductivity and increases electrical resistance. This, in turn, causes increased sparking density at the electrode surface and significantly more intense ablation energy and ablation rate as compared to the otherwise similar conditions shown in FIG. 6A. Such conditions provide for increased ablative capability, allowing the electrode and generated plasma to cut through, vaporize, or ablate adjacent tissue 68 at a significantly greater rate than possible in the configuration shown in FIG. 6A. For example, in an embodiment, the rate at which one may ablate tissue may increase by at least about 10%, at least about 20%, at least about 35%, at least about 50%, at least about 75%, at least about 100%, or at least about 200%.

FIGS. 6A and 6B also show how protrusion 58 aids in ensuring that a gap is advantageously present between the surface of electrode 50 and tissue 68. Such a protrusion 58 may comprise an electrically insulative material (e.g., a ceramic), or may in another embodiment comprise a portion of the electrode 50 (e.g., formed of metal).

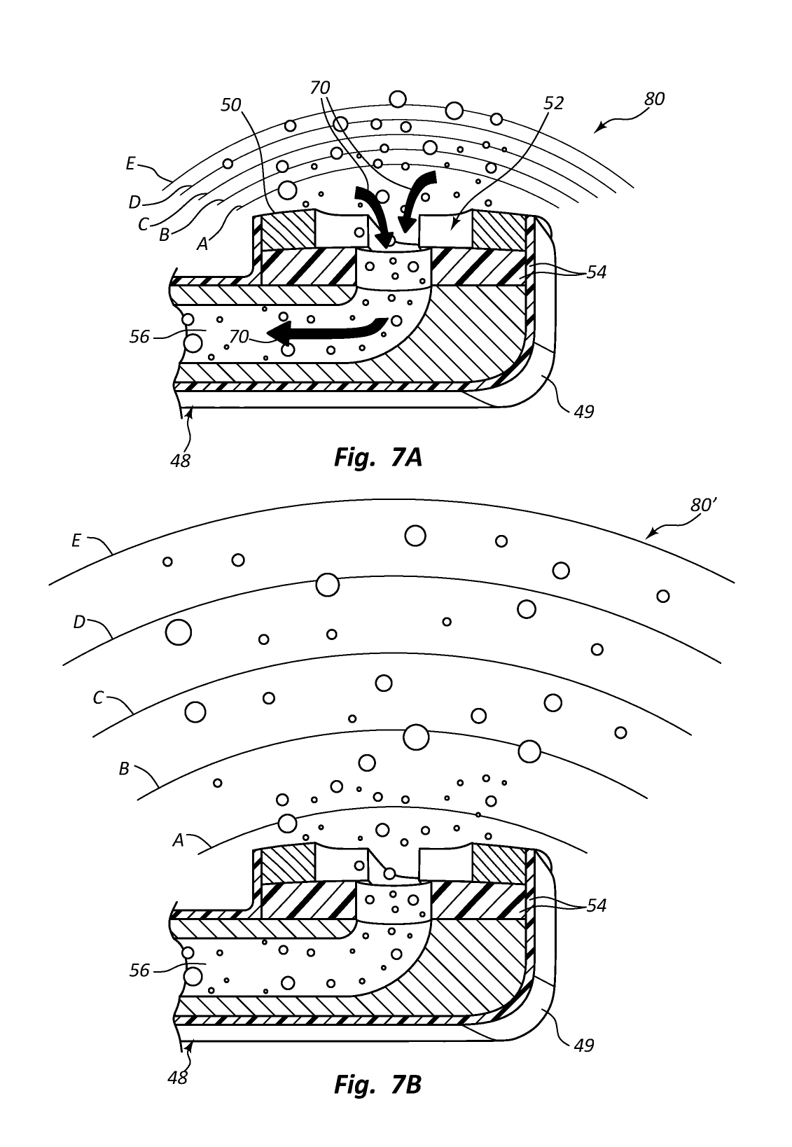

FIGS. 7A-7B schematically illustrate a radiant heat or energy field associated with operation in a normal ablation mode, where normal aspiration is provided, as compared to the heat or energy field associated with operation in a boosted ablation mode, where aspiration is temporarily restricted. As described above, when operating in a normal ablation mode, fluid (e.g., saline) is aspirated over the electrode surface (e.g., designated by arrows 70), providing active cooling of the electrode 50. During the normal ablation mode, as represented by FIG. 7A, the electrode generates an ablative sparking energy field 80 and associated temperature gradient characteristics. Various temperature gradient contour lines corresponding to decreasing temperatures as one moves from immediately adjacent the surface of electrode are labeled A, B, C, D, E, etc. For example, the area immediately adjacent to the electrode surface is at a given temperature, which is the hottest within field 80 (e.g., perhaps 500° C. or more). A temperature gradient of given characteristics is present, as the temperature drops as one moves further from the electrode, through gradient contour lines A, B, C, D, and E. For example, beyond contour line E, the temperature may be sufficiently low (e.g., 100° C. or lower) that ablation does not occur.

Because of cooling activity provided by aspiration, whether desired or not, cooling saline irrigant (e.g., initially at about 25-40° C.) is constantly being drawn through the energy field, causing energy field 80 to be compacted relative to how it would appear if no active fluid flow 70 were present. In other words, the temperature gradient is relatively steep, the contour lines A-E associated with given decreasing temperatures are relatively close together, and the associated size of ablative sparking energy field 80 is relatively small.

Upon restricting aspiration, as represented by FIG. 7B, active cooling is temporarily slowed or halted, and the temperature gradient associated with the region surrounding the electrode becomes significantly less steep, and the ablative sparking energy field 80′ generated by the electrode under these conditions is significantly larger. In other words, the energy field almost immediately expands as a result of the change in cooling conditions. The temperature gradient contour lines A-E are significantly further apart, resulting in a significantly larger energy field 80′ as compared to energy field 80. In addition, in at least some instances, the area immediately adjacent to the electrode may typically be at a temperature that is higher than the temperature associated with the normal ablation mode (e.g., at least 25% higher, at least 50% higher, at least 100% higher, or at least 250% higher).

While described in the context of embodiments where the boosted ablative mode is entered by temporarily restricting aspiration and reducing active cooling, it will be appreciated that another embodiment may provide a boost to ablation (although perhaps less in degree) by reducing the degree of any applied suction, rather than completely eliminating it altogether. For example, suction may be reduced by at least 50%, at least 75%, at least 80%, at least 90%, or at least 95%.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

What is claimed is:

1. An electrosurgical instrument for selectively operating in normal and boosted ablation modes, comprising:
an elongate probe having a handle portion and a distal end;
an electrode disposed at the distal end configured to ablate tissue;
an aspiration lumen through the elongate probe with an opening at the distal end so as to aspirate fluid;
a user-operable control component that, when actuated, places the electrosurgical instrument in normal ablation mode, with power delivered to the electrode and fluid aspiration by the aspiration lumen; and
a user-operable control component disposed on the handle portion that, when actuated, places the electrosurgical instrument in boosted ablation mode, with power delivered to the electrode, restricted or no fluid aspiration by the aspiration lumen to reduce or eliminate active cooling of the electrode and increase ablative sparking density at the electrode.

2. An electrosurgical instrument as in claim 1, wherein the user-operable control component disposed on the handle portion is configured to increase ablative sparking density at the electrode by at least 10% compared to when operating in normal ablation mode.

3. An electrosurgical instrument as in claim 1, wherein the user-operable control component disposed on the handle portion is configured to increase ablative sparking density at the electrode by at least 20% compared to when operating in normal ablation mode.

4. An electrosurgical instrument as in claim 1, wherein the user-operable control component disposed on the handle portion is configured to increase ablative sparking density at the electrode by at least 35% compared to when operating in normal ablation mode.

5. An electrosurgical instrument as in claim 1, wherein the user-operable control component disposed on the handle portion is configured to increase ablative sparking density at the electrode by at least 50% compared to when operating in normal ablation mode.

6. An electrosurgical instrument as in claim 1, wherein the user-operable control component for selectively placing the electrosurgical instrument in normal ablation mode and the user-operable control component disposed on the handle portion for selectively placing the electrosurgical instrument in boosted ablation mode are both provided by the user-operable control component disposed on the handle portion.

7. An electrosurgical instrument as in claim 6, wherein the user-operable control component disposed on the handle portion is configured to place the electrosurgical instrument in normal ablation mode when actuated a first time and, when actuated a second time, place the electrosurgical instrument in boosted ablation mode with increased ablative sparking density.

8. An electrosurgical instrument as in claim 1, further comprising at least one user-operable control component for selectively placing the electrosurgical instrument in coagulation mode.

9. An electrosurgical instrument as in claim 1, wherein the electrosurgical instrument includes a first user-operable control component for selectively placing the electrosurgical instrument in an ablation mode and a second user-operable control component for selectively placing the electrosurgical instrument in a coagulation mode.

10. An electrosurgical instrument as in claim 1, wherein the user-operable control component for selectively placing the electrosurgical instrument in normal ablation mode and the user-operable control component disposed on the handle portion for selectively placing the electrosurgical instrument in boosted ablation mode are separately actuated user-operable control components.

11. An electrosurgical instrument as in claim 1, wherein the user-operable control component disposed on the handle portion comprises a spring loaded switch that, when actively depressed while the electrosurgical instrument is operating in normal ablation mode, switches the electrosurgical instrument into boosted ablation mode by restricting or cutting off fluid aspiration by the aspiration lumen, thereby decreasing or eliminating active cooling and increasing ablative sparking density at the electrode, and, when released, increases fluid aspiration by the aspiration lumen and/or cuts off power to the electrode.

12. An electrosurgical instrument as in claim 1, wherein the user-operable control component disposed on the handle portion comprises a spring loaded switch that, when actively depressed while the aspiration lumen is aspirating fluid and the electrosurgical instrument is not operating in an ablation mode, places the electrosurgical instrument into boosted ablation mode by restricting or cutting off fluid aspiration and initiating delivery of power to the electrode, thereby providing increased ablative sparking density at the electrode, and, when released, increases fluid aspiration by the aspiration lumen and/or cuts off power to the electrode.

13. An electrosurgical instrument as in claim 1, wherein the user operable control component disposed on the handle portion comprises a toggle switch that, when actuated a first time, switches the electrosurgical instrument from one of coagulation mode or normal ablation mode to boosted ablation mode and, when actuated a second time, switches the electrosurgical instrument from boosted ablation mode to one of normal ablation mode, coagulation mode, or a deactivated mode.

14. An electrosurgical instrument as in claim 1, wherein a given amount of power up to 400 Watts is provided to the electrode independent of whether the electrosurgical instrument is operating in normal ablation mode or boosted ablation mode such that the ablative sparking density at the electrode is increased by reducing or cutting off aspiration by the aspiration lumen, not by increasing power to the electrode.

15. An electrosurgical instrument as in claim 1, wherein the opening of the aspiration lumen is positioned through the electrode.

16. An electrosurgical instrument as in claim 1, wherein a width of the opening of the aspiration lumen is less than a width of the aspiration lumen adjacent to the opening.

17. An electrosurgical instrument as in claim 1, wherein the geometry of the opening of the aspiration lumen is cross-shaped to provide sharp edges in the geometry of the electrode through which the opening of the aspiration lumen is disposed.

18. An electrosurgical instrument as in claim 1, wherein the electrosurgical instrument is configured for monopolar operation.

19. An electrosurgical instrument as in claim 1, wherein the electrosurgical instrument is configured for bipolar operation.

20. An electrosurgical instrument for selectively operating in normal ablation mode or boosted ablation mode, comprising:
an elongate probe having a handle portion and a distal end;
an electrode disposed at the distal end and configured to ablate tissue;
an aspiration lumen through the elongate probe with an opening at the distal end so as to aspirate fluid;
a first user-operable control component that, when actuated, places the electrosurgical instrument in normal ablation mode, with power delivered to the electrode and fluid aspiration by the aspiration lumen; and
a second user-operable control component, separate from the first user-operable control component and comprising a first button disposed on the handle portion, that, when actuated, places the electrosurgical instrument in boosted ablation mode, with power delivered to the electrode, restricted or no fluid aspiration by the aspiration lumen to reduce or eliminate active cooling of the electrode and increase ablative sparking density at the electrode.

21. An electrosurgical instrument as in claim 20, further comprising a third user-operable control component for placing the electrosurgical instrument in coagulation mode.

22. An electrosurgical instrument as in claim 21, wherein the first user-operable control component for placing the electrosurgical instrument in normal ablation mode comprises a first foot pedal remote from the handle portion, and the third user-operable control component for placing the electrosurgical instrument in coagulation mode comprises a second foot pedal remote from the handle portion.

23. An electrosurgical instrument as in claim 20, wherein the first user-operable control component comprises a second button disposed on the handle portion.

24. An electrosurgical instrument as in claim 23, wherein the second button is disposed on a top surface of the handle portion and the first button is disposed on a bottom surface of the handle portion.

25. An electrosurgical instrument as in claim 23, wherein the electrosurgical instrument is configured so that actuating only the second button places the electrosurgical device in normal ablation mode.

26. An electrosurgical instrument as in claim 25, wherein the second button is a toggle switch.

27. An electrosurgical instrument as in claim 25, wherein the second button is a safety switch that can only be actuated by being continuously depressed.

28. An electrosurgical instrument as in claim 23, wherein the electrosurgical instrument is configured so that actuating only the first button places the electrosurgical device in boosted ablation mode.

29. An electrosurgical instrument as in claim 28, wherein the first button is a safety switch that can only be actuated by being continuously depressed.

30. An electrosurgical instrument as in claim 23, wherein the electrosurgical instrument is configured so that the first and second buttons must both be actuated to place the electrosurgical device in boosted ablation mode.

31. An electrosurgical instrument as in claim 30, wherein the first button is a safety switch that can only be actuated by being continuously depressed and the second button is a toggle switch.

32. An electrosurgical instrument as in claim 30, wherein the first and second buttons are both safety switches that can only be actuated by being continuously depressed.

33. An electrosurgical instrument as in claim 20, wherein the electrosurgical instrument is configured so that actuation of the first user-operable control component places the electrosurgical instrument in normal ablation mode by initiating delivery of power to the electrode and initiating or continuing aspiration of fluid by the aspiration lumen.

34. An electrosurgical instrument as in claim 33, wherein the electrosurgical instrument is configured so that actuation of the second user-operable control component places the electrosurgical instrument in boosted ablation mode by restricting or cutting off aspiration of fluid by the aspiration lumen and continuing delivery of power to the electrode after the first user-operable control component has been actuated.

35. An electrosurgical instrument as in claim 33, wherein the electrosurgical instrument is configured so that actuation of the second user-operable control component places the electrosurgical instrument in boosted ablation mode by restricting or cutting off aspiration of fluid by the aspiration lumen and initiating delivery of power to the electrode when the first user-operable control component has not been actuated.

36. A method for ablating tissue, comprising:
providing an elongate electrosurgical instrument comprising a handle portion, a distal end, an electrode at the distal end, and an aspiration lumen;
positioning the electrode at a surgical site of a patient;
operating the electrosurgical instrument in normal ablation mode while aspirating fluid by the aspiration lumen; and
operating the electrosurgical instrument in boosted ablation mode by actuating a user-operable control component disposed on the handle portion to restrict aspiration of fluid through the aspiration lumen, reduce active cooling of the electrode, and increase ablative sparking density at the electrode compared to normal ablation mode.

37. A method as in claim 36, wherein operating the electrosurgical instrument in normal ablation mode is initiated by actuating a second user-operable control component in order to initiate delivery of power to the electrode and initiate or continue aspiration of fluid by the aspiration lumen.

38. A method as in claim 37, wherein the electrosurgical instrument is switched from normal ablation mode to boosted ablation mode by actuating the user-operable control component disposed on the handle portion in order to continue delivery of power to the electrode and restrict or cut off aspiration of fluid by the aspiration lumen.

39. A method as in claim 37, wherein operating the electrosurgical instrument in boosted ablation mode is initiated by actuating the user-operable control component disposed on the handle portion in order to initiate delivery of power to the electrode and restrict or cut off aspiration of fluid by the aspiration lumen.

40. A method as in claim 37, wherein operating the electrosurgical instrument in boosted ablation mode is initiated by actuating the user-operable control component disposed on the handle portion in order to initiate delivery of power to the electrode without initiating aspiration of fluid by the aspiration lumen.

41. A method as in claim 36, wherein operating the electrosurgical instrument in boosted ablation mode increases the ablative sparking density at the electrode by at least about 10% compared to normal ablation mode.

42. A method as in claim 36, wherein operating the electrosurgical instrument in boosted ablation mode increases the ablative sparking density at the electrode by at least about 20% compared to normal ablation mode.

43. A method as in claim 36, wherein operating the electrosurgical instrument in boosted ablation mode increases the ablative sparking density at the electrode by at least about 35% compared to normal ablation mode.

44. A method as in claim 36, wherein operating the electrosurgical instrument in boosted ablation mode increases the ablative sparking density at the electrode by at least about 50% compared to normal ablation mode.

45. A method as in claim 36, wherein operating the electrosurgical instrument in boosted ablation mode increases the rate of tissue ablation by at least about 10% compared to normal ablation mode.

46. A method as in claim 36, wherein operating the electrosurgical instrument in boosted ablation mode increases the rate of tissue ablation by at least about 20% compared to normal ablation mode.

47. A method as in claim 36, wherein operating the electrosurgical instrument in boosted ablation mode increases the rate of tissue ablation by at least about 35% compared to normal ablation mode.

48. A method as in claim 36, wherein operating the electrosurgical instrument in boosted ablation mode increases the rate of tissue ablation by at least about 50% compared to normal ablation mode.
